(19) United States
(12) Patent Application Publication
(10) Pub. No.: US 2022/0143194 A1
(43) Pub. Date: May 12, 2022

(54) SELENIUM ANTIBODY CONJUGATES

(71) Applicant: Regeneron Pharmaceuticals, Inc., Tarrytown, NY (US)

(72) Inventor: Thomas Nittoli, Pearl River, NY (US)

(21) Appl. No.: 17/454,186

(22) Filed: Nov. 9, 2021

Related U.S. Application Data
(60) Provisional application No. 63/112,044, filed on Nov. 10, 2020.

(51) Int. Cl. A61K 47/54; A61K 47/68

(52) U.S. Cl. CPC A61K 47/545; A61K 47/6803

(57) ABSTRACT

Provided herein are antibody conjugates, including antibody drug conjugates, that include selenium-containing linkers.

### RELATED APPLICATION

This application claims benefit of priority to U.S. Provisional Patent Application No. 63/112,044, filed Nov. 10, 2020, the contents of which are incorporated herein by reference in their entirety.

### FIELD

Provided herein are antibody conjugates, including antibody drug conjugates (ADCs), that include selenium-containing linkers.

### BACKGROUND

ADCs combine the power of antibody specificity with the ability to site specifically target a particular type of cell or tissue with a payload. Research in this area has drawn significant interest and has led to marketed pharmaceutical products, including ADCETRIS® (brentuximab vedotin) and KADCYLA™ (ado-trastuzumab emtansine). In many cases, conjugation of an antibody with a payload has been performed in a nonspecific manner whereby the payload is not conjugated to a defined position on the antibody, resulting in mixtures of ADCs that are difficult to purify. Additionally, standard conjugation methods result in variability in drug to antibody ratio (DAR), adding further complexity to the resulting ADC mixtures.

More recently, processes have been developed to site-specifically conjugate an antibody with a payload. See, e.g., Agarwal et al. Bioconjugate Chem. 2015, 26, 176-192. However, these processes have limitations, including lack of compatibility with certain payloads and/or chemical cross-reactivity. For example, Dennler et al. (Bioconjugate Chem. 2014, 25, 569-578) developed an SAc thiol linker (C6-SAc), but concluded that inefficient deacetylation of the intermediate thiol prevented the method from being entirely successful.

Thus, there is a continuing need for efficient, site-specific methods for producing ADCs and for ADCs produced by such methods.

### SUMMARY

Provided herein are ADCs having a selenium-containing linker that links an antigen-binding domain and a payload. In one embodiment, the antigen-binding domain is an antibody or antigen-binding fragment thereof. In another embodiment, the payload is a therapeutic agent or an imaging agent. In another embodiment, the therapeutic agent is a cytotoxin. In another embodiment, the ADCs provided herein have Formula I:

Z-(Gln-NH-L-Se-L¹-R)ₙ

or a pharmaceutically acceptable salt thereof, wherein:

Z is an antigen-binding domain;

Gln is a glutamine of the antigen-binding domain;

NH is the side chain NH of the Gln;

L and L¹ are the same or different and are each a linker;

R is a payload; and

n is an integer from 1 to 10.

In another embodiment, the ADCs provided herein are useful in methods of treatment or methods of imaging or diagnosis.

### BRIEF DESCRIPTION OF THE DRAWINGS

FIG. 1 is a depiction of a process for synthesizing ADCs provided herein, where Z is an antibody, L is ethylene; L¹ results from mc-vc-PAB; and R is MMAE.

FIGS. 2A and 2B show mass spectrometry spectra for deglycosylated mAb1 (degly-mAb1), deglycosylated mAb1-Se (degly-mAb1-1, following reaction with selenocystamine and MTGase), deglycosylated mAb1-Se-MMAE (degly-mAb1-1-mc-vc-PAB-MMAE, following reaction with mc-vc-PAB-MMAE), and a mock control reaction of mAb1 with mc-vc-PAB-MMAE.

FIG. 3 shows mass spectrometry spectra for deglycosylated ISOmAb (degly-ISOmAb), deglycosylated ISOmAb-Se (degly-ISOmAb-1, following reaction with selenocystamine and MTGase), and deglycosylated ISOmAb-Se-MMAE (degly-ISOmAb-1-mc-vc-PAB-MMAE, following reaction with mc-vc-PAB-MMAE).

FIG. 4 shows size-exclusion HPLC (SEC) for deglycosylated mAb1-Se-MMAE (degly-mAb1-1-mc-vc-PAB-MMAE) and deglycosylated ISOmAb-Se-MMAE (degly-ISOmAb-1-mc-vc-PAB-MMAE).

FIG. 5 shows cell viability in HER+ (SKBR3) and HER2− (H1975) cell lines when incubated with ADCs provided herein.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, “antigen-binding domain” means any peptide, polypeptide, nucleic acid molecule, scaffold-type molecule, peptide display molecule, or polypeptide-containing construct that is capable of specifically binding a particular antigen of interest. As used herein, “antigen-binding domain” includes antibodies and antigen-binding fragments of antibodies. All references to proteins, polypeptides and protein fragments herein are intended to refer to the human version of the respective protein, polypeptide or protein fragment unless explicitly specified as being from a non-human species.

As used herein, the term “specifically binds” or the like means that the antigen-binding domain forms a complex with a particular antigen characterized by a dissociation constant (K_{D}) of 500 pM or less, and does not bind other unrelated antigens under ordinary test conditions.

As used herein, “unrelated antigens” are proteins, peptides or polypeptides that have less than 95% amino acid identity to one another.

The term “antibody,” as used herein, means any antigen-binding molecule or molecular complex comprising at least one complementarity determining region (CDR) that specifically binds to or interacts with a particular antigen (e.g., human HER2). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

As used herein, the term “antigen-binding fragment” of an antibody means any naturally occurring, enzymatically obtainable, synthetic, or genetically engineered polypeptide or glycoprotein that specifically binds an antigen to form a complex.

As used herein, the term “human antibody” means antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

As used herein, the term “recombinant human antibody”, means all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences.

As used herein in the context of amino acid sequences, the term “substantial identity” or “substantially identical” means that two amino acid sequences, when optimally aligned, such as by the programs GAP or BESTFIT using default gap weights, share at least 95%, 98% or 99% sequence identity.

As used herein, the term “surface plasmon resonance”, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

As used herein, the term “K_{D}”, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

As used herein, pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates.

As used herein, treatment means any manner in which one or more of the symptoms of a disease or disorder are ameliorated or otherwise beneficially altered. Treatment also encompasses any pharmaceutical use of the compositions herein, such as use for treating tumors, including HER2 positive tumors, such as breast cancer.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, the IC₅₀ refers to an amount, concentration or dosage of a particular test compound that achieves a 50% inhibition of a maximal response in an assay that measures such response.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical. The term “alkylene” by itself or as part of another substituent means a divalent radical derived from an alkyl. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms. Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds. The term “alkenylene” by itself or as part of another substituent means a divalent radical derived from an alkenyl. Typically, an alkenyl (or alkenylene) group will have from 1 to 24 carbon atoms, including those groups having 10 or fewer carbon atoms. A “lower alkenyl” or “lower alkenylene” is a shorter chain alkenyl or alkenylene group, generally having six or fewer carbon atoms. Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom in the chain selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CHs, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂- and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. The terms “cycloalkylene” and “heterocycloalkylene” by themselves or as part of another substituent means a divalent radical derived from a cycloalkyl or heterocycloalkyl. Examples of cycloalkyl include, but are not limited to, cyclopentyl, cyclohexyl, 1-cyclohexenyl, 3-cyclohexenyl, cycloheptyl, norbornanyl, bicyclo(2.2.2)octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo(2.2.2)octanyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. The terms “arylene” and “heteroarylene” by themselves or as part of another substituent means a divalent radical derived from a aryl or heteroaryl. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The term “heteroarylium” refers to a heteroaryl group that is positively charged on one or more of the heteroatoms.

Each of the above terms are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R′″, —OC(O)R′, —C(O)R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″ C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “oxo” as used herein means an oxygen atom that is double bonded to a carbon atom.

As used herein, the term “heteroatom” or “ring heteroatom” is meant to include oxygen (O), nitrogen (N), sulfur (S), phosphorus (P), and silicon (Si).

Certain ADCs, including L and L¹, provided herein possess asymmetric carbon atoms (optical centers) or double bonds; the racemates, diastereomers, tautomers, geometric isomers and individual isomers are encompassed within the scope of the present disclosure. The ADCs provided herein do not include those which are known in the art to be too unstable to synthesize and/or isolate.

### II. ADCs for Use in Compositions and Methods

In one embodiment, provided herein are ADCs for use in the compositions and methods provided herein having Formula I:

Z-(Gln-NH-L-Se-L¹-R)ₙ

or a pharmaceutically acceptable salt thereof, wherein:

Z is an antigen-binding domain;

Gln is a glutamine of the antigen-binding domain

NH is the side chain NH of the Gln;

L and L¹ are the same or different and are each a linker;

R is a payload; and

n is an integer from 1 to 8.

In another embodiment, n is an integer from 1 to 6. In another embodiment, n is an integer from 1 to 4. In another embodiment, n is 2 or 4. In another embodiment, n is 2.

A. Antigen-Binding Domains Z

In one embodiment, antigen-binding domains, i.e., Z in Formula I, for use in the ADCs provided herein include any molecule that specifically interacts with a particular antigen. In certain embodiments, Z is an antibody or antigen-binding fragment of an antibody. In another embodiment, Z is an antibody. In another embodiment, Z is an antibody that comprises a glutamine residue. Antibodies comprising glutamine residues can be isolated from natural sources or engineered to comprise one or more glutamine residues. Techniques for engineering glutamine residues into an antibody polypeptide chain (glutaminyl-modified antibodies) are within the skill of the practitioners in the art. In other embodiments, Z is an N297Q mutant antibody. In further embodiments, Z is an antibody that has one or more engineered LLQG, LLQGG, LLQLLQG, LLQYQG, LLQGA, LLQGSG, SLLQG, LQG, LLQLQ, LLQLLQ, LLQGR, LLQYQGA, LQGG, LGQG or LLQLLQGA sites. See, e.g., U.S. Pat. No. 9,676,871 and U.S. Patent Application Publication No. 2003/0138785. In certain embodiments, the antibody is aglycosylated. In certain embodiments, Z is an antibody that is a monoclonal antibody, human antibody, humanized antibody, camelised antibody, or chimeric antibody. In other embodiments, Z is an antibody of any isotype (e.g., IgG, IgE, IgM, IgD, IgA and IgY), class (e.g., IgG1, IgG2, IgG3, IgG4, IgA1 and IgA2) or subclass. In some embodiments, Z has a molecular weight of at least 500, 600, 700, 800, 900, 1000, 10000, 50000 or 100000 Daltons.

In other embodiments, antigen-binding domains that can be used in the ADCs provided herein include antibodies, antigen-binding fragments of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., (see, e.g., Boersma and Pluckthun, 2011, Curr. Opin. Biotechnol. 22:849-857, and references cited therein)), and aptamers or portions thereof.

Methods for determining whether two molecules specifically bind one another are well known in the art and include, for example, equilibrium dialysis, surface plasmon resonance, and the like. For example, an antigen-binding domain, as used herein, includes polypeptides that bind a particular antigen (e.g., a target molecule (T) or an internalizing effector protein (E)) or a portion thereof with a K_{D} of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

In certain embodiments, the framework regions (FRs) of the antibodies or antigen-binding fragment thereof for use in the ADCs provided herein may be identical to the human germline sequences, or may be naturally or artificially modified. An amino acid consensus sequence may be defined based on a side-by-side analysis of two or more CDRs.

Methods and techniques for identifying CDRs within HCVR and LCVR amino acid sequences are well known in the art and can be used to identify CDRs. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The antigen-binding domains for use in the ADCs provided herein may comprise or consist of antigen-binding fragments of full antibody molecules. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments for use in the ADCs provided herein include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. In other embodiments, an antigen-binding fragment of an antibody includes other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains.

In certain embodiments, an antigen-binding fragment of an antibody will comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody for use in the ADCs provided herein include: (i) V_{H}-C_{H}1; (ii) V_{H}—C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; V_{L}—C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2- C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In further embodiments, an antigen-binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In another embodiment, the antigen-binding domains used in the ADCs provided herein may comprise or consist of human antibodies and/or recombinant human antibodies, or antigen-binding fragments thereof.

In another embodiment, the antigen-binding domains used in the ADCs provided herein may comprise or consist of recombinant human antibodies or antigen-binding fragments thereof. In one embodiment, such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In another embodiment, the antigen-binding domains used in the ADCs provided herein also include bispecific antigen-binding molecules, such as bispecific antibodies. Methods for making bispecific antibodies are known in the art and may be used to construct bispecific antigen-binding molecules for use herein. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4:6, 1-11, and references cited therein, for a review of the foregoing formats). See also, e.g., US2018/0134794, which discloses bispecific antigen-binding molecules. Briefly, bispecific antigen binding molecules may comprise a first antigen-binding domain (also referred to herein as “D1”), and a second antigen-binding domain (also referred to herein as “D2”). The simultaneous binding of the two separate epitopes by the bispecific antigen-binding molecule results in effective ligand blocking with minimal activation of target signaling. In certain embodiments, D1 and D2 domains of a bispecific antibody are non-competitive with one another. Non-competition between D1 and D2 means that, the respective monospecific antigen binding proteins from which D1 and D2 were derived do not compete with one another for binding to the target. Exemplary antigen-binding protein competition assays are known in the art. In certain embodiments, D1 and D2 bind to different (e.g., non-overlapping, or partially overlapping) epitopes on the target. Bispecific antigen-binding molecules may be constructed using the antigen-binding domains of two separate monospecific antibodies. For example, a collection of monoclonal monospecific antibodies may be produced using standard methods known in the art. The individual antibodies thus produced may be tested pairwise against one another for cross-competition to the target protein. If two different antibodies are able to bind to the target at the same time (i.e., do not compete with one another), then the antigen-binding domain from the first antibody and the antigen-binding domain from the second, non-competitive antibody can be engineered into a single bispecific antibody. A bispecific antigen-binding molecule can be a single multifunctional polypeptide, or it can be a multimeric complex of two or more polypeptides that are covalently or non-covalently associated with one another. Any antigen binding construct which has the ability to simultaneously bind two separate, non-identical epitopes of the target molecule is regarded as a bispecific antigen-binding molecule. Bispecific antigen-binding molecules, or variants thereof, may be constructed using standard molecular biological techniques (e.g., recombinant DNA and protein expression technology) as will be known to a person of skill in the art. In another embodiment, bispecific antibodies are also provided wherein one arm of the bispecific antibody binds to an epitope on a first target protein, and the other arm of the bispecific antibody binds to a second epitope on a second target protein. Other exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. (Epub: Dec. 4, 2012)).

In another embodiment, the antigen binding domains for use in the ADCs provided herein also include antibodies comprising variants of any of the HCVR, LCVR, and/or CDR amino acid sequences known in the art. In one embodiment, variants include variants of any of the HCVR, LCVR, and/or CDR amino acid sequences known in the art having one or more conservative substitutions. For example, the antigen binding domains include antibodies or antigen binding fragments thereof having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences known in the art. In another embodiment, the antigen binding domains include antibodies or antigen binding fragments thereof also include variants having substantial sequence identity to any of the HCVR, LCVR, and/or CDR amino acid sequences known in the art. In certain embodiments, residue positions which are not identical differ by conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In one embodiment, conservative amino acids substitution groups are: valine-leucine-isoleucine, phenylalanine-tyrosine, lysine-arginine, alanine-valine, glutamate-aspartate, and asparagine-glutamine. In another embodiment, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence identity between two different amino acid sequences is typically measured using sequence analysis software. Sequence analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another algorithm when comparing a sequence provided herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

i. Epitope Mapping and Related Technologies

The epitope to which the antigen-binding domains bind may consist of a single contiguous sequence of 3 or more (e.g., 3, 4, 5, 6, 7, 8, 9, 10, 11, 12, 13, 14, 15, 16, 17, 18, 19, 20 or more) amino acids of a target protein. Alternatively, the relevant epitope may consist of a plurality of non-contiguous amino acids (or amino acid sequences) of the target protein. In some embodiments, the epitope is located on or near the binding domain of the target protein. In other embodiments, the epitope is located outside of the binding domain of the target protein.

Various techniques known to persons of ordinary skill in the art can be used to determine the epitope with which the antigen-binding domains used in the ADCs provided herein interact. Exemplary techniques that can be used to determine an epitope or binding domain of a particular antigen-binding domain include, e.g., point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain interacts is hydrogen/deuterium exchange detected by mass spectrometry. In general terms, the hydrogen/deuterium exchange method involves deuterium-labeling the protein of interest, followed by binding the antigen-binding domain to the deuterium-labeled protein. Next, the protein/antigen-binding domain complex is transferred to water to allow hydrogen-deuterium exchange to occur at all residues except for the residues protected by the antigen-binding domain (which remain deuterium-labeled). After dissociation of the antigen-binding domain, the target protein is subjected to protease cleavage and mass spectrometry analysis, thereby revealing the deuterium-labeled residues which correspond to the specific amino acids with which the antigen-binding domain interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antigen-binding domain interacts.

ii. Preparation of Human Antibodies

In one embodiment, the antibodies for use in the ADCs provided herein are fully human antibodies. Methods for generating monoclonal antibodies, including fully human monoclonal antibodies are known in the art. Any such known methods can be used in the context of the present disclosure to make human antibodies that specifically bind to a human protein target.

Using VELOCIMMUNE™ technology, for example, or any other similar known method for generating fully human monoclonal antibodies, high affinity chimeric antibodies to a human protein target are initially isolated having a human variable region and a mouse constant region. The antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human antibody. While the constant region selected may vary according to specific use, high affinity antigen-binding and target specificity characteristics reside in the variable region. In certain instances, fully human antibodies are isolated directly from antigen-positive B cells.

iii. Bioequivalents

The antigen-binding domains for use in the ADCs provided herein encompass proteins having amino acid sequences that vary from those of the described antibodies but that retain the ability to bind the target proteins. Such variant antigen-binding domains comprise one or more additions, deletions, or substitutions of amino acids when compared to parent sequence, but exhibit biological activity that is essentially equivalent to that of the described antibodies.

Two antigen-binding domains are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding domains will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding domains are bioequivalent if there are no clinically meaningful differences in their safety, purity, and potency.

In one embodiment, two antigen-binding domains are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding domains are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antigen-binding domain or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antigen-binding domain (or its target) is measured as a function of time; and (d) in a well-controlled clinical trial that establishes safety, efficacy, or bioavailability or bioequivalence of an antigen-binding domain.

Bioequivalent variants of antigen-binding domains for use in the ADCs provided herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding domains may include variants comprising amino acid changes which modify the glycosylation characteristics of the antigen-binding domain, e.g., mutations which eliminate or remove glycosylation.

iv. Species Selectivity and Species Cross-Reactivity

In certain embodiments, the antigen-binding domains for use in the ADCs provided herein bind to a human target protein but not to target protein from other species. In other embodiments, the antigen-binding domains for use in the ADCs provided herein bind to a human target protein and to a target protein from one or more non-human species. For example, the antigen-binding domains for use in the ADCs provided herein may bind to a human target protein and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee target protein. In one embodiment, the antigen-binding domains specifically bind human target protein and cynomolgus monkey (e.g., Macaca fascicularis) target protein. In other embodiments, antigen-binding domains for use herein bind human target protein but do not bind, or bind only weakly, to cynomolgus monkey target protein.

v. Exemplary Antibodies and Antigen Targets

Antibodies for use in the ADCs provided herein can have binding specificity for any antigen (target protein) deemed suitable to those of skill in the art. In certain embodiments, the antigen is a transmembrane molecule (e.g., receptor). In one embodiment, the antigen is expressed on a tumor. In some embodiments, the binding agents interact with or bind to tumor antigens, including antigens specific for a type of tumor or antigens that are shared, overexpressed, or modified on a particular type of tumor. In one embodiment, the antigen is expressed on solid tumors. Exemplary antigens include, but are not limited to, lipoproteins; alphal-antitrypsin; a cytotoxic T-lymphocyte associated antigen (CTLA), such as CTLA-4; vascular endothelial growth factor (VEGF); receptors for hormones or growth factors; protein A or D; fibroblast growth factor receptor 2 (FGFR2), EpCAM, GD3, FLT3, PSMA, PSCA, MUC1, MUC16, STEAP, STEAP2, CEA, TENB2, EphA receptors, EphB receptors, folate receptor, FOLRI, mesothelin, cripto, alphavbeta6, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044; erythropoietin; osteoinductive factors; immunotoxins; a bone morphogenetic protein (BMP); T-cell receptors; surface membrane proteins; integrins, such as CD11a, CD11 b, CD11c, CD18, an ICAM, VLA-4 and VCAM; a tumor associated antigen such as AFP, ALK, B7H4, BAGE proteins, β-catenin, brc-abl, BRCA1, BORIS, CA9 (carbonic anhydrase IX), caspase-8, BCMA, SLAMF7, GPNMB, UPK3A, CD20, CD40, CD123, CDK4, CEA, CLEC12A, c-kit, cMET, CTLA4, cyclin-B1, CYP1B1, EGFR, EGFRvIII, endoglin, Epcam, EphA2, ErbB2/Her2, ErbB3/Her3, ErbB4/Her4, ETV6-AML, Fra-1, FOLR1, GAGE proteins, GD2, GD3, GloboH, glypican-3, GM3, gp100, Her2, HLA/B-raf, HLA/EBNA1, HLA/k-ras, HLA/MAGE-A3, hTERT, IGF1R, LGRS, LMP2, MAGE proteins, MART-1, mesothelin, ML-IAP, Muc1, Muc16, CA-125, MUM1, NA17, NGEP, NY-BR1, NY-BR62, NY-BR85, NY-ESO1, OX40, p15, p53, PAP, PAX3, PAXS, PCTA-1, PDGFR-α, PDGFR-β, PDGF-A, PDGF-B, PDGF-C, PDGF-D, PLAC1, PRLR, PRAME, PSCA, PSGR, PSMA (FOLH1), RAGE proteins, Ras, RGSS, Rho, SART-1, SART-3, Steap-1, Steap-2, STn, survivin, TAG-72, TGF-β, TMPRSS2, Tn, TNFRSF17, TRP-1, TRP-2, tyrosinase, and uroplakin-3, and fragments of any of the above-listed polypeptides; cell-surface expressed antigens; MUC16; c-MET; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2). In some embodiments, the antigen is PRLR or HER2. In some embodiments, the antigen is HER2. In some embodiments, the antigen is human HER2. In some embodiments, the antigen is STEAP2. In some embodiments the antigen is human STEAP2. In some examples, the MAGE proteins are selected from MAGE-1, -2, -3, -4, -6, and -12. In some examples, the GAGE proteins are selected from GAGE-1 and GAGE-2.

In certain embodiments, the antibody comprises a glutamine residue at one or more heavy chain positions numbered 295 in the EU numbering system. In the present disclosure, this position is referred to as glutamine 295, or as Gln295, or as Q295. Those of skill in the art will recognize that this is a conserved glutamine residue in the wild type sequence of many antibodies. In other embodiments, the antibody can be engineered to comprise a glutamine residue. In certain embodiments, the antibody comprises one or more N297Q mutations. Techniques for modifying an antibody sequence to include a glutamine residue are within the skill of those in the art (see, e.g., Ausubel et al. Current Protoc. Mol. Biol. (John Wiley & Sons)).

B. Payloads R

In one embodiment, R is a therapeutic agent or an imaging agent. In another embodiment, R is a therapeutic agent such as a cytotoxic agent, a chemotherapeutic drug, or a radioisotope. In other embodiments, R is a positron emitter and/or a chelating moiety. In other embodiments, payloads for use in the ADCs provided herein include cytotoxins, anti-inflammatory agents, steroids, glucocorticoids, LXR modulators, anti-viral agents and antibiotics. In another embodiment, payloads for use in the ADCs provided herein are small molecule compounds, e.g., compounds that have a molecular weight less than 2000 Daltons, less than 1500 Daltons, less than 1000 Daltons, less than 750 Daltons or less than 500 Daltons.

Cytotoxic agents for use as a payload herein include any agent that is detrimental to the growth, viability or propagation of cells, including, but not limited to, tubulin-interacting agents and DNA-damaging agents. Examples of suitable cytotoxic agents and chemotherapeutic agents that can be used in the ADCs provided herein include, e.g., 1-(2-chloroethyl)-1,2-dimethanesulfonyl hydrazide, 1,8-dihydroxy-bicyclo(7.3.1)trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin γ₁), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. In certain embodiments, the cytotoxic agent used as a payload in the ADCs provided herein is a maytansinoid such as DM1 or DM4, a tomaymycin derivative, or a dolastatin derivative. In other embodiments, the cytotoxic agent used as a payload in the ADCs provided herein is an auristatin such as MMAE, MMAF, or derivatives thereof. In one embodiment, the cytotoxic agent used as a payload in the ADCs provided herein is MMAE (monomethyl auristatin E):

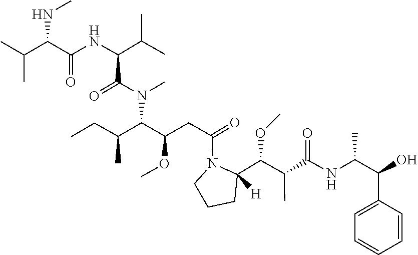 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Other cytotoxic agents known in the art are contemplated for use as payloads in the ADCs provided herein, including, e.g., protein toxins such ricin, C. difficile toxin, pseudomonas exotoxin, diphtheria toxin, botulinum toxin, bryodin, saporin, pokeweed toxins (i.e., phytolaccatoxin and phytolaccigenin), and others such as those set forth in Sapra et al., Pharmacol. & Therapeutics, 2013, 138:452-469. In some embodiments, the payload is a maytansinoid described in US 2019/0151323, US 2016/0375147, U.S. Pat. No. 9,950,076, or U.S. Pat. No. 10,570,151; or a tubulysin described in WO 2020/132658.

In certain embodiments, the cytotoxic agent for use in the ADCs provided herein is a maytansinoid, e.g., derivative of maytansine. Suitable maytansinoids include DM1, DM4, or derivatives, stereoisomers, or isotopologues thereof. Suitable maytansinoids also include those disclosed in WO 2014/145090A1, WO 2015/031396A1, US 2016/0375147A1, and US 2017/0209591A1.

In some embodiments, the maytansinoid has the following structure:

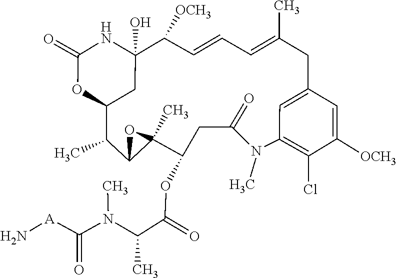

wherein A is an optionally substituted arylene or heteroarylene.

In some embodiments, the maytansinoid has the following structure:

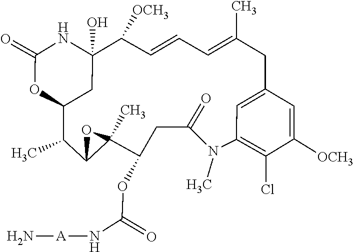

wherein A is an optionally substituted arylene or heteroarylene.

In some embodiments, the maytansinoid has the following structure:

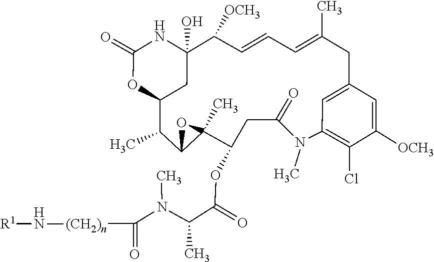

wherein n is an integer from 1-12 and R¹ is alkyl.

In some embodiments, the maytansinoid is:

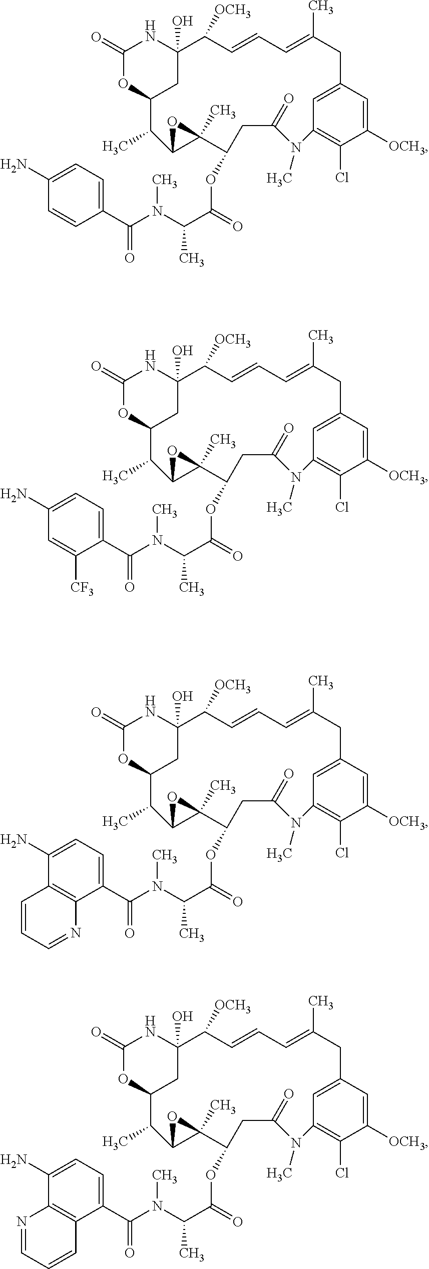

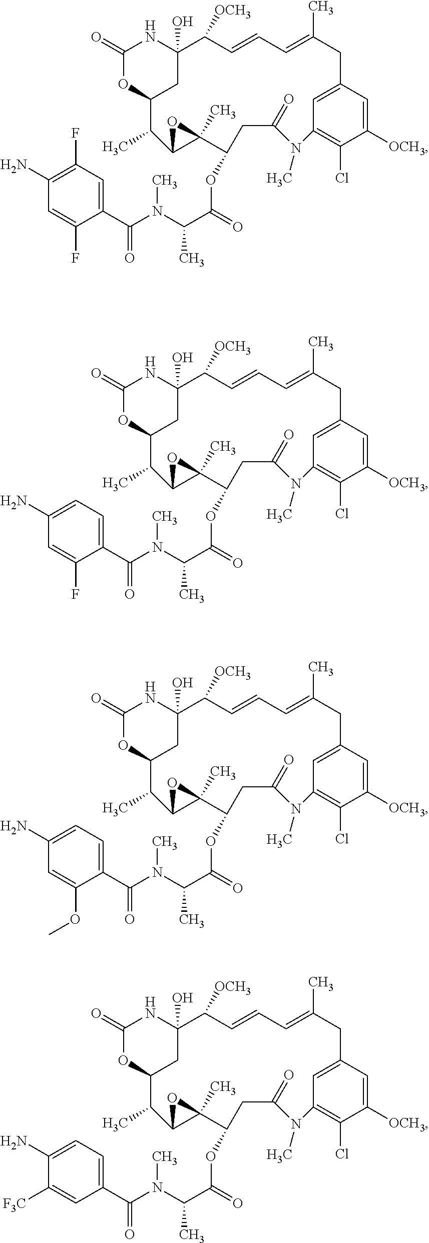

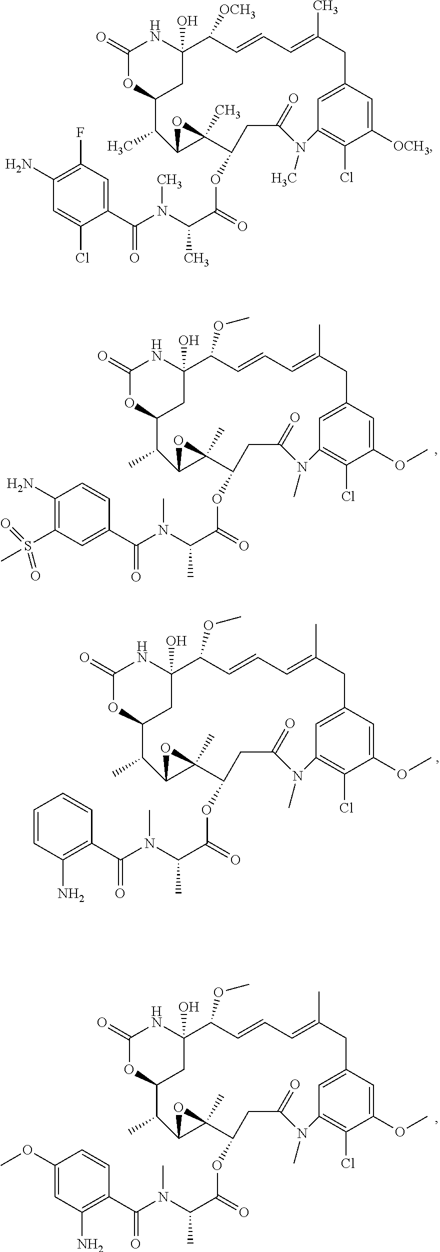

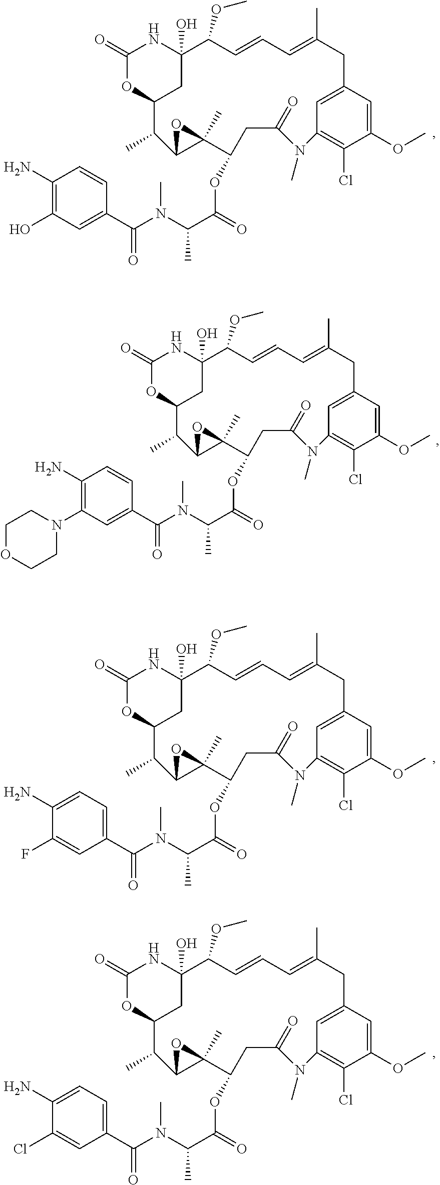

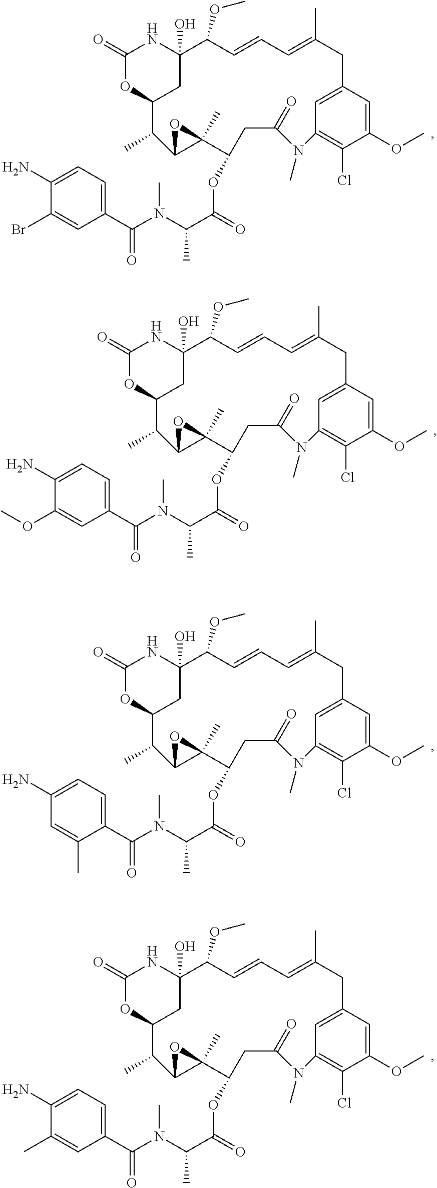

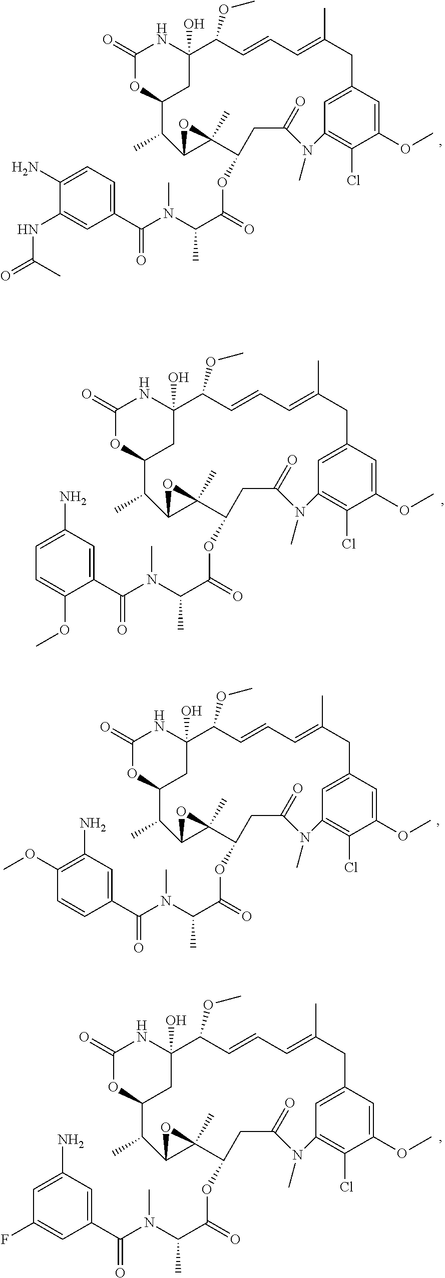

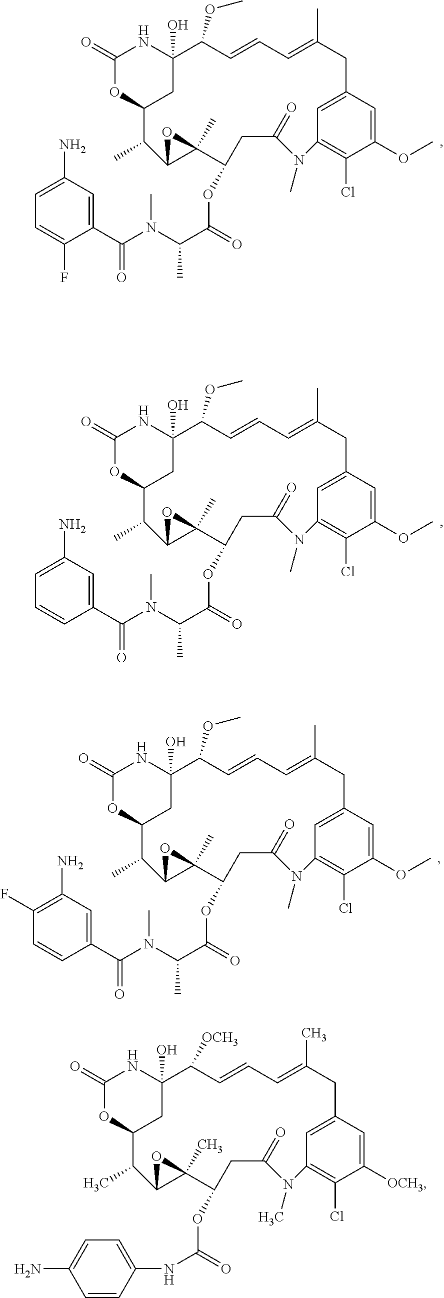

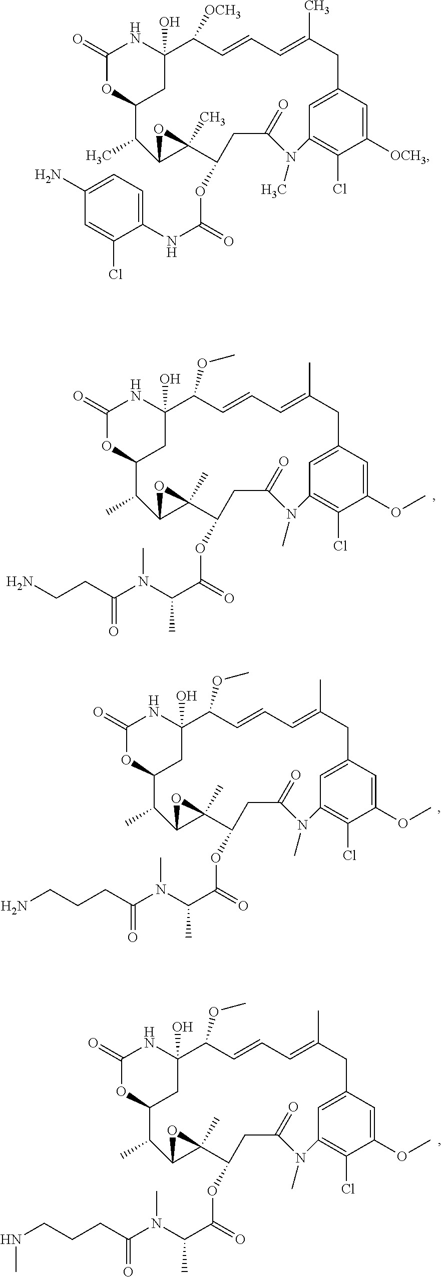

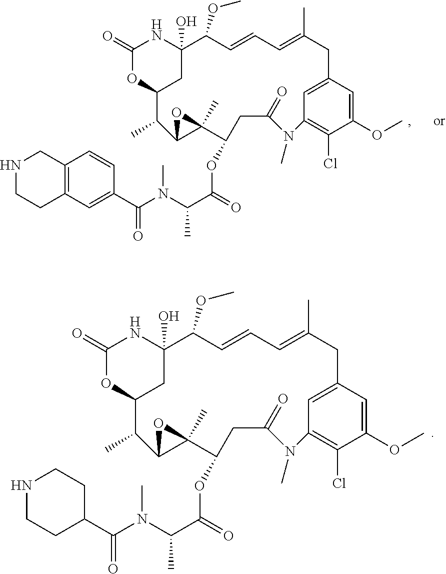 [SMILES: COc1cc2cc(c1Cl)N(C)C(=O)C[C@H](OC(=O)[C@H](C)N(C)C(=O)c1ccc3c(c1)CCNC3)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)C1CCNCC1)[C@]1(C)O[C@H]1[C@H](C)[C@@H]1C[C@@](O)(NC(=O)O1)[C@H](OC)/C=C/C=C(\C)C2]

In some embodiments, the maytansinoid is:

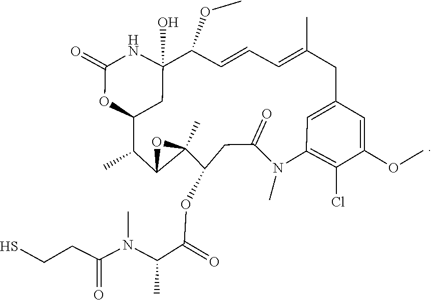 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

In some embodiments, the maytansinoid is:

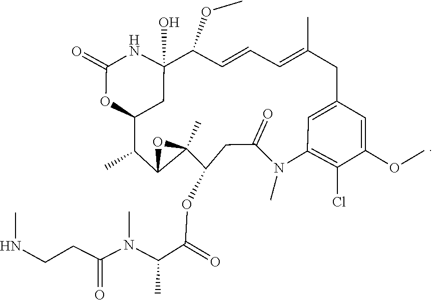 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In other embodiments, R is a radionuclide. In these embodiments, the ADCs provided herein are antibody-radionuclide conjugates (ARCs). Radionuclides that can be used as payloads herein include, e.g., ²²⁵Ac, ²¹²Bi, ²¹³Bi, ¹³¹I, ¹⁸⁶Re, ²²⁷Th, ²²²Rn, ²²³Ra, ²²⁴Ra, and ⁹⁰Y.

In certain embodiments, ADCs provided herein are those wherein an antigen binding domain is conjugated to a linker-drug composition as set forth in International Patent Publication No. WO2014/145090, e.g., compound “7,” depicted below:

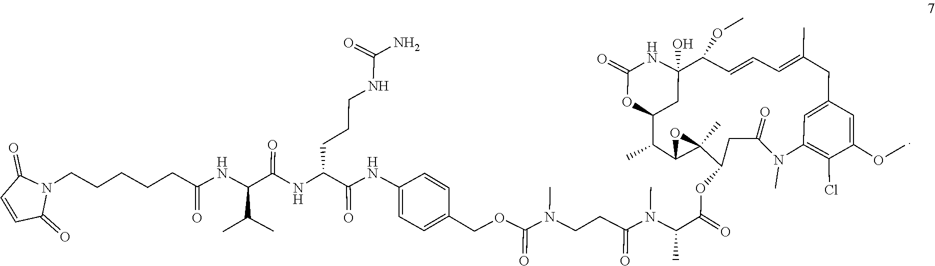 [SMILES: COc1cc2cc(c1Cl)N(C)C(=O)C[C@H](OC(=O)[C@H](C)N(C)C(=O)CCN(C)C(=O)OCc1ccc(NC(=O)[C@@H](CCCNC(N)=O)NC(=O)[C@H](NC(=O)CCCCCN3C(=O)C=CC3=O)C(C)C)cc1)[C@]1(C)O[C@H]1[C@H](C)[C@@H]1C[C@@](O)(NC(=O)O1)[C@H](OC)/C=C/C=C(\C)C2]

Also provided herein are ADCs comprising an antigen binding domain conjugated to a cytotoxic agent. In certain embodiments, the cytotoxic agent is a maytansinoid. In certain embodiments, the maytansinoid is a compound having the following formula:

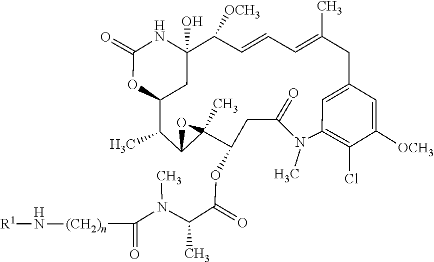

wherein n is an integer from 1-12 and R¹ is alkyl. In certain embodiments, the maytansinoid is

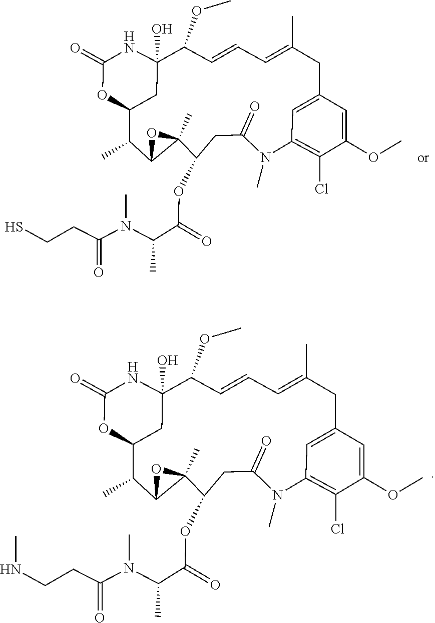 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2 ; CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the cytotoxic agent is a maytansinoid, and the maytansinoid is covalently attached to the antigen binding domain via non-cleavable linker. In certain embodiments, the cytotoxic agent is a maytansinoid, and the maytansinoid is covalently attached to the antigen binding domain via cleavable linker.

In another embodiment, the cytotoxic agent is a tubulysin. See, e.g., International Patent Publication No. WO 2020/132658. In another embodiment, the cytotoxic agent has the formula:

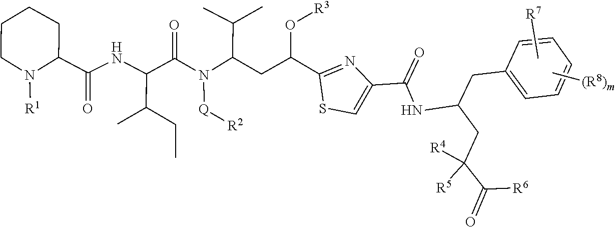

where
R¹ is C₁-C₁₀ alkyl;
R³ is —C(O)C₁-C₅ alkyl, —C(O)N(H)C₁-C₁₀ alkyl, or —(C₁-C₁₀ alkylene)-NR^{3a}R^{3b},

wherein R^{3a} and R^{3b} are independently in each instance, hydrogen, alkyl, alkenyl, alkynyl, cycloalkyl, aryl, heteroaryl, and acyl, wherein alkyl, alkenyl, alkynyl, cycloalkyl, aryl, heteroaryl, and acyl are optionally substituted;

R⁴ and R⁵ are, independently in each instance, hydrogen or C₁-C₅ alkyl;
R⁶ is —OH or —NHNH₂;
R⁷ is, independently in each instance, hydrogen, —OH, halogen, or —NR^{7a}R^{7b},

wherein R^{7a} and R^{7b} are independently in each instance, hydrogen, alkyl, alkenyl, alkynyl, cycloalkyl, aryl, heteroaryl, acyl, and amino acid residue, wherein alkyl, alkenyl, alkynyl, cycloalkyl, aryl, heteroaryl, and acyl are optionally substituted;

R⁸ is, independently in each instance, hydrogen, deuterium, —NHR⁹, or halogen,

wherein R⁹ is hydrogen, —C₁-C₅ alkyl, or —C(O)C₁-C₅ alkyl; and

m is 1 or 2;

Q is —CH₂— or —O— wherein

when Q is —O—, then R² is C₁-C₁₀ alkyl, C₁-C₁₀ alkynyl,
—C₁-C₁₀ alkylene-(5-membered heteroaryl), —C₁-C₃ alkylene-Q¹-(CH₂)ₙaryl, or C₁-C₃ hydroxyalkyl; or

when Q is —CH₂—, then R² is C₅-C₁₀ alkyl, C₁-C₁₀ alkynyl,
—C₁-C₁₀ alkylene-(5-membered heteroaryl), —C₁-C₃ alkylene-Q¹-(CH₂)ₙaryl, or C₁-C₃ hydroxyalkyl; and
   Q¹ is —CH₂— or —O—;
   wherein said heteroaryl is unsubstituted or substituted with alkyl, aminoalkyl, hydroxylalkyl, carboxyalkyl, benzyl, or phenyl;
   wherein said aryl is unsubstituted or substituted with nitro or amino; and
   wherein n is an integer from 1 to 5.

In some embodiments, R is a steroid described in U.S. Pat. No. 10,711,032 or WO 2019/136487; a rifamycin analog described in WO 2020/132483; or an LXR modulator described in WO 2018/213082 or WO 2020/106780.

In other embodiments, R is a positron emitter and/or a chelating moiety. In one embodiment, positron emitters include those that form stable complexes with the chelating moiety and have physical half-lives suitable for immuno-PET imaging purposes. In certain embodiments, positron emitters include ⁸⁹Zr, ⁶⁸Ga, ⁶⁴Cu, ⁴⁴Sc, and ⁸⁶Y.

In certain embodiments, R includes a chelating moiety. Chelating moieties for use herein are chemical moieties that comprise a portion capable of chelating a positron emitter, i.e., capable of reacting with a positron emitter to form a coordinated chelate complex. In certain embodiments, chelating moieties include those that allow efficient loading of the particular metal and form metal-chelator complexes that are sufficiently stable in vivo for diagnostic uses, e.g., immuno-PET imaging. In another embodiment, chelating moieties include those that minimize dissociation of the positron emitter and accumulation in mineral bone, plasma proteins, and/or bone marrow depositing to an extent suitable for diagnostic uses.

Non-limiting examples of chelating moieties for use herein include those that form stable complexes with positron emitters ⁸⁹Zr, ⁶⁸Ga, ⁶⁴Cu, ⁴⁴Sc, and/or ⁸⁶Y. In other embodiments, chelating moieties include those described in Nature Protocols, 5(4): 739, 2010; Bioconjugate Chem., 26(12): 2579 (2015); Chem Commun (Camb), 51(12): 2301 (2015); s, 12: 2142 (2015); Mol. Imaging Biol., 18: 344 (2015); Eur. J. Nucl. Med. Mol. Imaging, 37:250 (2010); Eur. J. Nucl. Med. Mol. Imaging (2016). doi:10.1007/s00259-016-3499-x; Bioconjugate Chem., 26(12): 2579 (2015); WO 2015/140212A1; and U.S. Pat. No. 5,639,879.

In other embodiments, chelating moieties also include those that comprise desferrioxamine (DFO), 1,4,7,10-tetraazacyclododecane-1,4,7,10-tetraacetic acid (DOTA), diethylenetriaminepentaacetic acid (DTPA), ethylenediaminetetraacetic acid (EDTA), (1,4,7,10-Tetraazacyclododecane-1,4,7,10-tetra(methylene phosphonic) acid (DOTP), (1R, 4R, 7R, 10R)-a′a″a′″a″″-Tetramethyl-1,4,7,10-tetraazacyclododecane-1,4,7,10-tetraacetic acid (DOTMA), 1,4,8,11-Tetraazacyclotetradecane-1,4,8,11-tetraacetic acid (TETA), H₄octapa, H₆phospa, H₂dedpa, H₆decapa, H₂azapa, HOPO, DO2A, 1,4,7-triazacyclononane-N,N′,N″-triacetic acid (NOTA), 1,4,7,10-Tetrakis(carbamoylmethyl)-1,4,7,10-tetraazacyclododecane (DOTAM), 1,4,8,11-tetraazabicyclo[6.6.2]hexadecane-4,11-dicetic acid (CB-TE2A), 1,4,7,10-Tetraazacyclododecane (Cyclen), 1,4,8,11-Tetraazacyclotetradecane (Cyclam), octadentate chelators, hexadentate chelators, phosphonate-based chelators, macrocyclic chelators, chelators comprising macrocyclic terephthalamide ligands, bifunctional chelators, fusarinine C and fusarinine C derivative chelators, triacetylfusarinine C (TAFC), ferrioxamine E (FOXE), ferrioxamine B (FOXB), ferrichrome A (FCHA), and the like.

C. Linkers L and L¹

i. L

In certain embodiments, L is any group or moiety that links, connects, or bonds the selenium with the side chain NH of Gln. In one embodiment, L can be conjugated to one or more glutamine residues via transglutaminase-based chemo-enzymatic conjugation (see, e.g., Dennler et al., Bioconjugate Chem. 2014, 25, 569-578). For example, in the presence of transglutaminase, one or more glutamine residues of an antibody can be coupled to a primary amine compound, such as a primary amine compound containing selenium. In certain embodiments, the primary amine is a diselenide such as (H₂N-L-Se)₂. In some embodiments, L is any divalent group, including alkylene, alkenylene, cycloalkenylene, arylene, divalent polyethylene glycol (PEG) groups, and combinations thereof.

In certain embodiments, L has one of the following formulas:

(CH₂)ₙ;

(CH₂CH₂O)ₙ—(CH₂)ₚ;

(CH₂)ₙ—N(H)C(O)—(CH₂)ₘ;

(CH₂CH₂O)ₙ—N(H)C(O)—(CH₂CH₂O)ₘ—(CH₂)ₚ;

(CH₂)ₙ—C(O)N(H)—(CH₂)ₘ;

(CH₂CH₂O)ₙ—C(O)N(H)—(CH₂CH₂O)ₘ—(CH₂)ₚ;

(CH₂)ₙ—N(H)C(O)—(CH₂CH₂O)ₘ—(CH₂)ₚ;

(CH₂CH₂O)ₙ—N(H)C(O)—(CH₂)ₘ;

(CH₂)ₙ—C(O)N(H)—(CH₂CH₂O)ₘ—(CH₂)ₚ; and

(CH₂CH₂O)ₙ—C(O)N(H)—(CH₂)ₘ;

where n is an integer selected from 1 to 12;

m is an integer selected from 0 to 12; and

p is an integer selected from 0 to 2.

In another embodiment, L is alkylene. In another embodiment, L is ethylene.

ii. L¹

In certain embodiments, L¹ is any group or moiety that links, connects, or bonds the selenium with a payload. Suitable linkers may be found, for example, in Antibody-Drug Conjugates and Immunotoxins, Phillips, G. L., Ed.; Springer Verlag: New York, 2013; Antibody-Drug Conjugates, Ducry, L., Ed.; Humana Press, 2013; Antibody-Drug Conjugates, Wang, J., Shen, W.-C., and Zaro, J. L., Eds.; Springer International Publishing, 2015. In certain embodiments, the L¹ group for the ADCs provided herein is sufficiently stable to exploit the circulating half-life of the antigen binding domain and, at the same time, capable of releasing its payload after antigen-mediated internalization of the ADC. Linker L¹ can be cleavable or non-cleavable. Cleavable linkers for use as L¹ herein include linkers that are cleaved by intracellular metabolism following internalization, e.g., cleavage via hydrolysis, reduction, or enzymatic reaction. Non-cleavable linkers for use as L¹ herein include linkers that release an attached payload via lysosomal degradation of the antigen binding domain following internalization. Suitable L¹ linkers include, but are not limited to, acid-labile linkers, hydrolysis-labile linkers, enzymatically cleavable linkers, reduction labile linkers, self-immolative linkers, and non-cleavable linkers. Suitable L¹ linkers also include, but are not limited to, those that are or comprise peptides, carbohydrates, glucuronides, polyethylene glycol (PEG) units, hydrazones, mal-caproyl units, dipeptide units, valine-citruline units, and para-aminobenzyl (PAB) units.

Any linker molecule or linker technology known in the art can be used as L¹ to create or construct an ADC provided herein. In certain embodiments, the L¹ linker is a cleavable linker. In other embodiments, the L¹ linker is a non-cleavable linker. In certain embodiments, L¹ linkers that can be used in the ADCs provided herein include linkers that comprise or consist of e.g., MC (6-maleimidocaproyl), MP (maleimidopropanoyl), val-cit (valine-citrulline), val-ala (valine-alanine), dipeptide site in protease-cleavable linkers, ala-phe (alanine-phenylalanine), dipeptide site in protease-cleavable linkers, PAB (p-aminobenzyloxycarbonyl), and variants and combinations thereof. Additional examples of L¹ linkers that can be used in the ADCs provided herein are disclosed, e.g., in U.S. Pat. No. 7,754,681 and in Ducry, Bioconjugate Chem., 2010, 21:5-13, and the references cited therein.

In certain embodiments, the L¹ linkers are stable in physiological conditions. In certain embodiments, the L¹ linkers are cleavable, for instance, able to release at least the payload portion in the presence of an enzyme or at a particular pH range or value. In some embodiments, an L¹ linker comprises an enzyme-cleavable moiety. In one embodiment, enzyme-cleavable L¹ linkers include, but are not limited to, peptide bonds, ester linkages, and hydrazones. In some embodiments, the L¹ linker comprises a cathepsin-cleavable linker.

In some embodiments, the L¹ linker comprises a non-cleavable moiety.

In some embodiments, the L¹ linker comprises one or more amino acids. Suitable amino acids include natural, non-natural, standard, non-standard, proteinogenic, non-proteinogenic, and L- or D-α-amino acids. In some embodiments, the L¹ linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the L¹ linker comprises lysine, valine, and citrulline. In some embodiments, the L¹ linker comprises lysine, valine, and alanine. In some embodiments, the L¹ linker comprises valine and alanine.

In some embodiments, the L¹ linker comprises a self-immolative group. The self-immolative group can be any such group known to those of skill in the art. In particular embodiments, the self-immolative group is p-aminobenzyl (PAB), or a derivative thereof. Useful derivatives include p-aminobenzyloxycarbonyl (PABC). Those of skill in the art will recognize that a self-immolative group is capable of carrying out a chemical reaction which releases the remaining atoms of an L¹ linker from a payload.

In other embodiments, the L¹ group can be modified with one or more enhancement groups. In certain embodiments, the enhancement group can be linked to the side chain of any amino acid in L¹. In one embodiment, amino acids for linking enhancement groups include lysine, asparagine, aspartate, glutamine, glutamate, and citrulline. The link to the enhancement group can be a direct bond to the amino acid side chain, or the link can be indirect via a spacer and/or reactive group. In one embodiment, spacers and reactive groups include any described herein. In certain embodiments, the enhancement group can be any group that imparts a beneficial effect to the payload, linker payload, or ADC including, but not limited to, biological, biochemical, synthetic, solubilizing, imaging, detecting, and reactivity effects, and the like. In certain embodiments, the enhancement group is a hydrophilic group. In certain embodiments, the enhancement group is a cyclodextrin. In certain embodiments, the enhancement group is an alkyl, heteroalkyl, alkenyl, heteroalkenyl sulfonic acid, heteroalkenyl taurine, heteroalkenyl phosphoric acid or phosphate, heteroalkenyl amine (e.g., quaternary amine), or heteroalkenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the enhancement group is capable of improving solubility of the remainder of the ADC. In certain embodiments, the alkyl, heteroalkyl, alkenyl, or heteroalkenyl sulfonic acid is substituted or non-substituted. In certain embodiments, the alkyl, heteroalkyl, alkenyl, or heteroalkenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkenyl sulfonic acid is —(CH₂)₁₋₅SO₃H. In another embodiment, the heteroalkyl or heteroalkenyl sulfonic acid is —(CH₂)ₙ—NH—(CH₂)₁₋₅SO₃H, wherein n is 1, 2, 3, 4, or 5. In another embodiment, the alkyl, heteroalkyl, alkenyl, or heteroalkenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkenyl, or heteroalkenyl sulfonic acid is —(CH₂CH₂O)ₙ—C(O)NH—(CH₂)₁₋₅SO₃H, wherein m is 1, 2, 3, 4, or 5. In another embodiment, the alkyl, heteroalkyl, alkenyl, or heteroalkenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkenyl, or heteroalkenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkenyl, or heteroalkenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

### III. Synthesis of the ADCs

Also provided herein is a method of synthesizing the ADCs provided herein. The method involves the step of reacting an antigen-binding domain containing glutamine with a diselenide of formula (H2N-L-Se)2 to provide a compound of formula II:

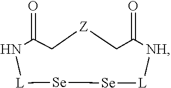

where Z is an antigen-binding domain;

each

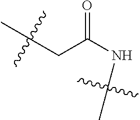 [SMILES: *CC(=O)N*]

is the side chain of Gln; and L is a linker. In one embodiment, the reaction of the antigen-binding domain and the diselenide is mediated by a transglutaminase enzyme. In another embodiment, the transglutaminase enzyme is a murine transglutaminase enzyme. In another embodiment, when the Gin of the antigen-binding domain is Q295 of an N297 antibody, then prior to reaction of the antigen-binding domain with the diselenide, the antigen-binding domain is reacted with a PNGase, such as without limitation PNGaseF, to deglycosylate N297.

In one embodiment, the compound of formula II is then reacted with a reducing agent at pH less than or equal to 6 to form a reduced diselenide. In another embodiment, the reaction of the compound of formula II with a reducing agent is performed at pH less than or equal to 5, or less than or equal to 4. In one embodiment, the reducing agent is tris(2-carboxyethyl)phosphine (TCEP). In another embodiment, the reduction of the diselenide is performed in the presence of L¹-R, wherein L¹ comprises a group that reacts with the reduced diselenide to form a covalent bond. In one embodiment, the group that reacts with the reduced diselenide is a maleimide group, an iodoacetamide group or a bromoacetamide group. In one embodiment, the group that reacts with the reduced diselenide is a maleimide group. In a further embodiment, the reduction of the compound of formula II in the presence of L¹-R produces an ADC provided herein.

Thus, in one embodiment, provided herein is a method of synthesizing the ADCs provided herein by optionally, when the Gln of Z-Gln is Q295 of an N297 antibody, then prior to step (a) the Z-Gln is reacted with a PNGaseF to deglycosylate N297; then

(a) reacting Z-Gln with (H₂N-L-Se)₂ and a murine transglutaminase to form a compound of formula II:

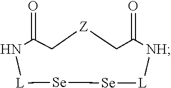

and

(b) reacting the product of step (a) with a reducing agent at pH less than or equal to 6 to form a reduced diselenide in the presence of L¹-R, wherein L¹ comprises a group that reacts with the reduced diselenide to form a covalent bond, thereby forming Z-Gln-NH-L-Se-L¹-R.

In another embodiment, provided herein is a method of synthesizing the ADCs provided herein by optionally, when the Gln of Z-Gln is Q295 of an N297 antibody, then prior to step (a) the Z-Gln is reacted with a PNGaseF to deglycosylate N297; then

(a) reacting Z-Gln with (H₂N-L-Se)₂ and a murine transglutaminase to form a compound of formula II:

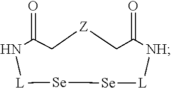

and

(b) reacting the product of step (a) with TCEP at pH less than or equal to 6 to form a reduced diselenide in the presence of L¹-R, wherein L¹ comprises a maleimide group that reacts with the reduced diselenide to form a covalent bond, thereby forming Z-Gln-NH-L-Se-L¹-R.

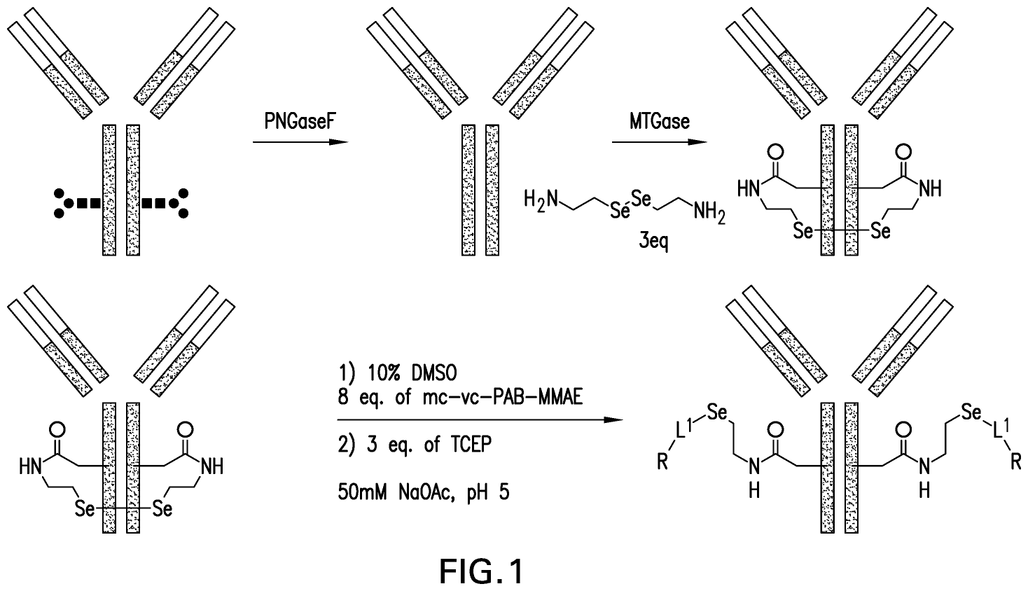

In another embodiment, provided herein is a method of synthesizing the ADCs provided herein according to the method shown in FIG. 1.

In another embodiment, provided herein is a compound prepared by the processes disclosed herein. In one embodiment, the compound prepared by a process disclosed herein is a compound of formula II:

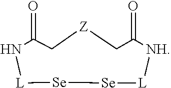

In another embodiment, the compound prepared by a process disclosed herein has formula IIa:

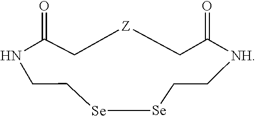

### IV. Pharmaceutical Compositions

The pharmaceutical compositions provided herein contain therapeutically effective amounts of one or more of ADCs provided herein and a pharmaceutically acceptable carrier.

The ADCs can be formulated into suitable pharmaceutical preparations. Typically, the ADCs described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more ADCs or pharmaceutically acceptable salts is (are) mixed with a suitable pharmaceutical carrier. In certain embodiments, the concentrations of the ADCs in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of an ADC is dissolved, suspended, dispersed or otherwise mixed in a selected carrier at an effective concentration such that the treated condition is relieved or ameliorated. Pharmaceutical carriers suitable for administration of the ADCs provided herein include any such carriers known to those skilled in the art to be suitable for the particular mode of administration.

In some embodiments, the ADC is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and well known to those of skill in the art, and then extrapolated therefrom for dosages for humans. In some embodiments, the ADC is administered in a method to achieve a therapeutically effective concentration of the payload. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the ADC in specific subjects or subject populations.

The concentration of ADC in the pharmaceutical composition will depend on absorption, tissue distribution, inactivation and excretion rates of the ADC, the physicochemical characteristics of the ADC, the dosage schedule, and amount administered as well as other factors known to those of skill in the art. For example, the amount that is delivered is sufficient to ameliorate one or more of the symptoms of a disease or disorder disclosed herein.

The compositions may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions.

The compositions may include other active compounds to obtain desired combinations of properties. The ADCs provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to herein. It is to be understood that such combination therapy constitutes a further aspect of the compositions and methods of treatment provided herein.

### V. Dosing

The compounds and pharmaceutical compositions provided herein may be dosed in certain therapeutically or prophylactically effective amounts, certain time intervals, certain dosage forms, and certain dosage administration methods as described below.

The methods provided herein encompass treating a patient regardless of subject's age, although some diseases or disorders are more common in certain age groups.

The ADC provided herein, or a pharmaceutically acceptable salt thereof, can be administered repeatedly if necessary, for example, until the subject experiences stable disease or regression, or until the subject experiences disease progression or unacceptable toxicity.

The ADC provided herein, or a pharmaceutically acceptable salt thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the ADC provided herein, or a pharmaceutically acceptable salt thereof, is administered once or more than once each day, for example, for a period of time. The term “continuous” is intended to mean that a therapeutic compound, such as the ADC provided herein, or a pharmaceutically acceptable salt thereof, is administered daily for an uninterrupted period of at least 10 days to 52 weeks. The term “intermittent” or “intermittently” as used herein is intended to mean stopping and starting at either regular or irregular intervals. For example, intermittent administration of the ADC provided herein, or a pharmaceutically acceptable salt thereof, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the ADC provided herein, or a pharmaceutically acceptable salt thereof, is administered daily or continuously but with a rest period. In some such embodiments, administration is once a day for two to six days, then a rest period with no administration for five to seven days.

### VI. Methods of Treatment

In another embodiment, a method of treating a subject with an ADC provided herein, or a pharmaceutically acceptable salt thereof, is provided. In another embodiment, a method of treating a subject with a pharmaceutical composition comprising an ADC provided herein, or a pharmaceutically acceptable salt thereof, is provided. The pharmaceutical composition comprises any of the ADCs disclosed herein, or a pharmaceutically acceptable salt thereof, and a pharmaceutically acceptable carrier.

The ADCs provided herein are useful, inter alia, for the treatment, prevention and/or amelioration of any disease or disorder associated with or mediated by expression, signaling or activity of the target protein of the antigen-binding domain.

In certain embodiments, where the payload is a cytotoxic agent, the ADCs provided herein may be used to treat primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the ADCs provided herein are used to treat one or more of the following cancers: acute myelogenous leukemia, adult T-cell leukemia, astrocytomas, bladder cancer, breast cancer, PRLR positive (PRLR+) breast cancer, cervical cancer, cholangiocarcinoma, chronic myeloid leukemia, colon cancer, colorectal cancer, endometrial cancer, esophageal cancer, gastric cancer, glioblastomata, head and neck cancer (e.g., head and neck squamous cell carcinoma (HNSCC)), Kaposi's sarcoma, kidney cancer, leiomyosarcomas, liver cancer, lung cancer (e.g., small cell lung cancer, non-small cell lung cancer (NSCLC)), lymphomas, malignant gliomas, malignant mesothelioma, melanoma, mesothelioma, malignant mesothelioma, MFH/fibrosarcoma, multiple myeloma, nasopharyngeal cancer, osteosarcoma, ovarian cancer, pancreatic carcinoma, prostate cancer, castrate-resistant prostate cancer, renal cell carcinoma, residual cancer wherein “residual cancer” means the existence or persistence of one or more cancerous cells in a subject following treatment with an anti-cancer therapy, rhabdomyosarcoma, small cell lung cancer, stomach cancer, synovial sarcoma, thyroid cancer, uterine cancer and Wilms' tumor. In some embodiments, the cancer is breast cancer. In some embodiments, the cancer is prostate cancer.

In the context of the methods of treatment provided herein, the ADCs may be administered as a monotherapy (i.e., as the only therapeutic agent) or in combination with one or more additional therapeutic agents (examples of which are described elsewhere herein).

### VII. Combination Therapy with a Second Active Agent

Provided herein are compositions comprising any of the ADCs provided herein in combination with one or more additional therapeutically active components, and methods of treatment comprising administering such combinations to a subject.

The ADCs provided herein may be co-formulated with and/or administered in combination with one or more additional therapeutically active component(s) selected from the group consisting of: a MET antagonist (e.g., an anti-MET antibody (e.g., onartuzumab, emibetuzumab, and H4H14639D) or small molecule inhibitor of MET), an EGFR antagonist (e.g., an anti-EGFR antibody (e.g., cetuximab or panitumumab) or small molecule inhibitor of EGFR (e.g., gefitinib or erlotinib)), an antagonist of another EGFR family member such as Her2/ErbB2, ErbB3 or ErbB4 (e.g., anti-ErbB2 (e.g., trastuzumab or T-DM1 {KADCYLA®}), anti-ErbB3 or anti-ErbB4 antibody or small molecule inhibitor of ErbB2, ErbB3 or ErbB4 activity), an antagonist of EGFRvIII (e.g., an anti-EGFRvIII antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody or small molecule kinase inhibitor such as, e.g., imatinib mesylate or sunitinib malate), a PDGF ligand inhibitor (e.g., anti-PDGF-A, -B, -C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354), an Ang2 antagonist (e.g., an anti-Ang2 antibody disclosed in US 2011/0027286 such as H1H685P), a FOLH1 antagonist (e.g., an anti-FOLH1 antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin (e.g., anti-UPK3A) antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGRS antagonist (e.g., an anti-LGRS antibody), a monovalent CD20 antagonist (e.g., a monovalent anti-CD20 antibody such as rituximab), a CD20×CD3 bispecific antibody, a PD-1 blocking agent (e.g., an anti-PD-1 antibody such as pembrolizumab or nivolumab), etc. Other agents that may be beneficially administered in combination with antibodies provided herein include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

Illustratively, a PD-1 inhibitor such as an anti-PD-1 antibody can be combined with an ADC as described herein.

In another embodiment, provided herein are pharmaceutical compositions comprising any of the ADCs provided herein in combination with one or more chemotherapeutic agents. Examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

The ADCs provided herein may also be administered and/or co-formulated in combination with antivirals, antibiotics, analgesics, corticosteroids, steroids, oxygen, antioxidants, COX inhibitors, cardioprotectants, metal chelators, IFN-gamma, and/or NSAIDs.

The additional therapeutically active component(s), e.g., any of the agents listed above or derivatives thereof, may be administered just prior to, concurrent with, or shortly after the administration of an ADC provided herein. In another embodiment, provided are pharmaceutical compositions in which an ADC provided herein is co-formulated with one or more of the additional therapeutically active component(s) as described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., an ADC provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

### VIII. Examples

The examples below are meant to illustrate certain embodiments provided herein, and not to limit the scope of this disclosure.

### Example 1

Antibody Deglycosylation

Two antibodies, an anti-HER2 antibody having variable regions derived from humAb4D5-8 from Carter et al., PNAS 1992, 89, 4285 (mAb1), and a non-binding isotype control derived from an immunological antigen having no relation to oncology (ISOmAb), were deglycosylated using 400 U/mg mAb of PNGaseF (NEB P0704L) in PBS pH 7.4 at 37° C. overnight. The reaction mixture was buffer exchanged to PBS pH 7.4 using spin filters (Amicon, 30 kDa cut-off). This allowed the 295Q residue to be accessed by the transglutaminase enzyme in Example 2 to conjugate the antibodies to a maximum loading of 2.

### Example 2

Bacterial Transglutaminase Conjugation of Selenocystamine

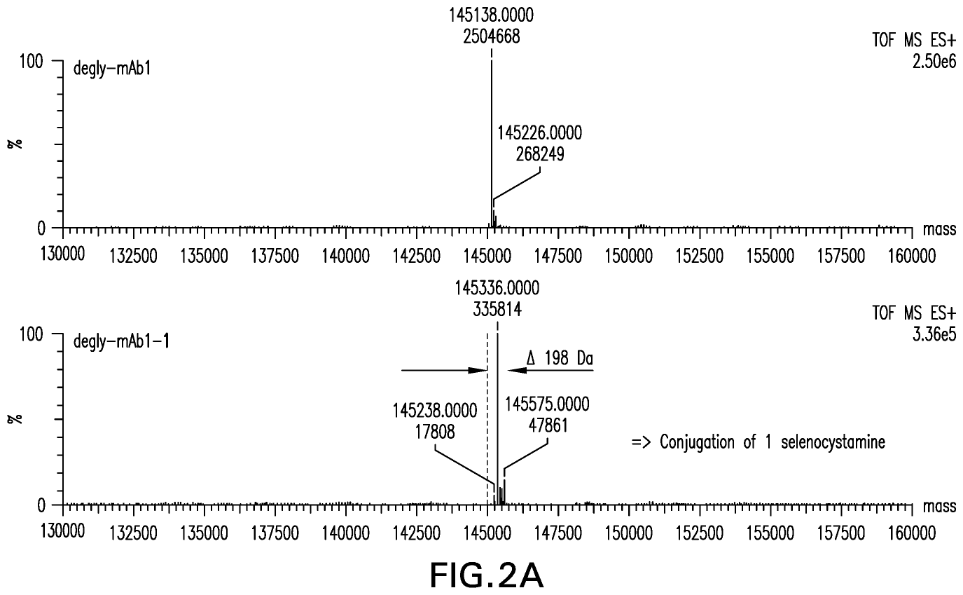

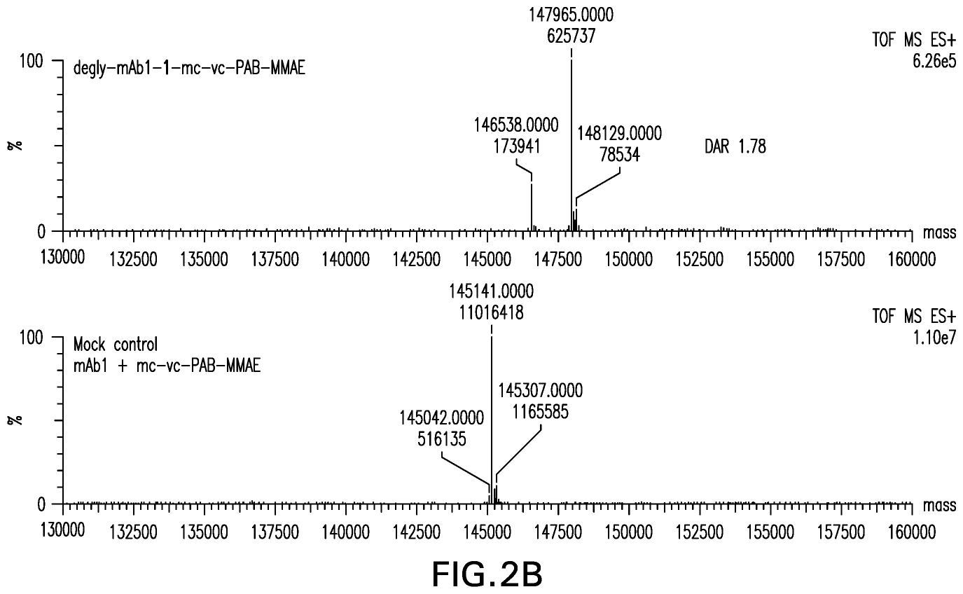

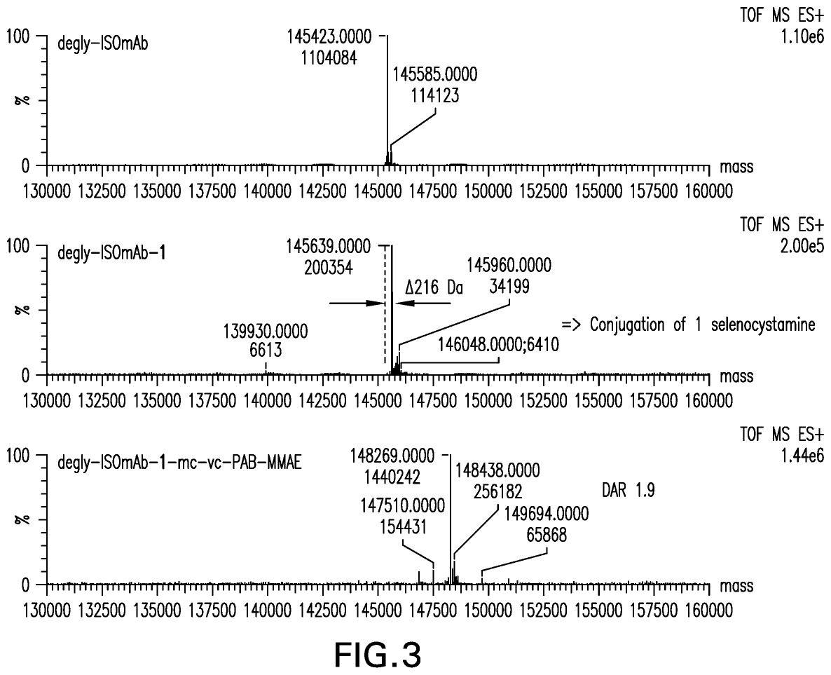

Deglycosylated ISOmAb (degly-ISOmAb) and deglycosylated mAb1 (degly-mAb1) antibodies (EXAMPLE 1) were conjugated at 1 mg/mL in PBS pH 7.4. Selenocystamine (compound 1, Catalog #S0520 Sigma-Aldrich) was added in a 3-fold molar excess over antibody and the enzymatic reaction was initiated by addition of 12 units of bacterial transglutaminase (Zedira, T1001) per mg antibody and incubated at 37° C. for 4-16 hours. The resulting conjugate was purified by ion exchange chromatography (GE Capto S ImpRes, using 20 mM NaOAc, pH 5 and 0 M NaCl to 0.5 M NaCl as gradient) and buffer exchanged to 20 mM NaOAc, pH 5. The conjugates (degly-ISOmAb-1 and delgy-mAb1-1) were analyzed by ESI-MS for the determination of the linker antibody ratio (LAR) using a Waters Acquity UPLC. The chromatographic separation was achieved on a C4 column (2.1×50 mm ACQUITY UPLC BEH protein C4, 1.7 μm, 300 Å) in a 10 min gradient (minute:percentage of mobile phase B; 0:10%, 1:10%, 5:90%, 7:90%, 7.2:10%, 10:10%). The mobile phase A was 0.1% formic acid in water and mobile phase B was 0.1% formic acid in acetonitrile. The flow rate was set at 0.3 mL/min. The detector time-of-flight (TOF) scan was set from m/z 500-4500 with major parameters as listed (Capillary voltage 3.0 kV; Sampling Cone 80V; Source Offset at 100V; Source temperatures 150° C.; Desolvation temperature 450° C.; Cone gas 0 L/hr; Desolvation gas 800 L/hr). The spectra were deconvoluted with MaxEnt function within MassLynx software. The resulting molecular ions which when weighted according to intensities corresponded to the loadings listed in Table 1. The actual mass spec spectra are listed in FIGS. 2A and 2B, and FIG. 3. Size-exclusion HPLC established that all conjugates were >95% monomeric (Table 3).

TABLE 1
Summary of intensity-weighted average linker loadings in ISOmAb
and mAb1 conjugates for selenocystamine conjugation.

| | | | |
|---|---|---|---|
| Molecular Ion MW | Corresponding | Relative | Intensity weighted |
| (Da) | linker loading | intensity | average loading |

degly-ISOmAb-1

| | | | |
|---|---|---|---|
| 145639 | 1 | 200354 | 1 |

delgy-mAb-1

| | | | |
|---|---|---|---|
| 145336 | 1 | 335814 | 1 |

### Example 3

Conjugating mc-VC-PAB-MMAE Linker Payload

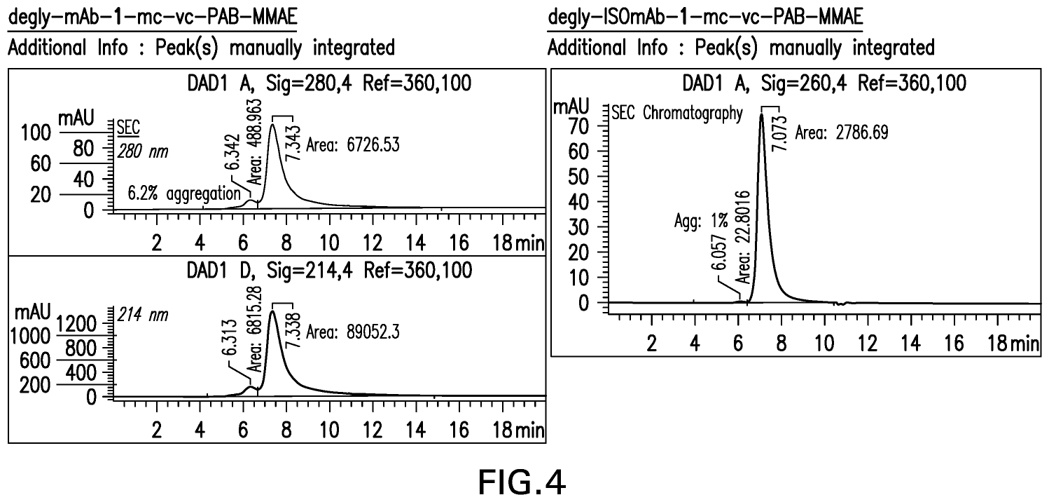

The selenocystamine-antibodies (EXAMPLE 2) were conjugated at 3-5 mg/mL in 50 mM NaOAc, pH 5 and 10% DMSO. The linker-payload (mc-VC-PAB-MMAE, Doronina, S. O. et al., Nat. Biotechnol., 2003, 21, 778) was added in an 12-fold molar excess over antibody, and tris(2-carboxyethyl)phosphine (TCEP) was added in an 3-fold molar excess over antibody. The reaction was incubated at room temperature for 1 hour. The conjugates were purified by Size-Exclusion Chromatography (SEC). Drug to antibody ratio (DAR) was determined by LC-MS (according to method described in EXAMPLE 2). The resulting molecular ions which when weighted according to intensities corresponded to the loadings listed in Table 2. The actual mass specrometry spectra are listed in FIGS. 2A and 2B, and FIG. 3. Size-exclusion HPLC (SEC) established that all conjugates were >93% monomeric (Table 3, FIG. 4).

TABLE 2
The summary of intensity-weighted average linker-
payload loadings in degly-ISOmAb-1-mc-vc-PAB-MMAE
and degly-mAb1-1-mc-vc-PAB-MMAE conjugates.

| | | | |
|---|---|---|---|
| Molecular Ion MW | Corresponding | Relative | Intensity weighted |
| (Da) | linker loading | intensity | average loading |

degly-ISOmAb-1-mc-vc-PAB-MMAE

| | | | |
|---|---|---|---|
| 146840 | 1 | 154431 | 1.90 |
| 148269 | 2 | 1440242 | |

degly-mAb1-1-mc-vc-PAB-MMAE

| | | | |
|---|---|---|---|
| 146538 | 1 | 173941 | 1.78 |
| 147965 | 2 | 625737 | |

TABLE 3
Purity (SEC) and DAR (ESI-MS)
of 1-mc-vc-PAB-MMAE conjugates.

| | Drug to Antibody | |
|---|---|---|
| | Ratio | Purity |
| Antibody Drug Conjugate | (ESI-MS DAR) | (SEC) |
| degly-ISOmAb-1-mc-vc-PAB-MMAE | 1.9 | 99.2% |
| degly-mAb1-1-mc-vc-PAB-MMAE | 1.8 | 93.2% |

### Example 4

In Vitro Cytotoxicity Assays

The ability of various antibody-drug conjugates and naked payloads to kill antigen-expressing tumor cells in vitro was assessed.

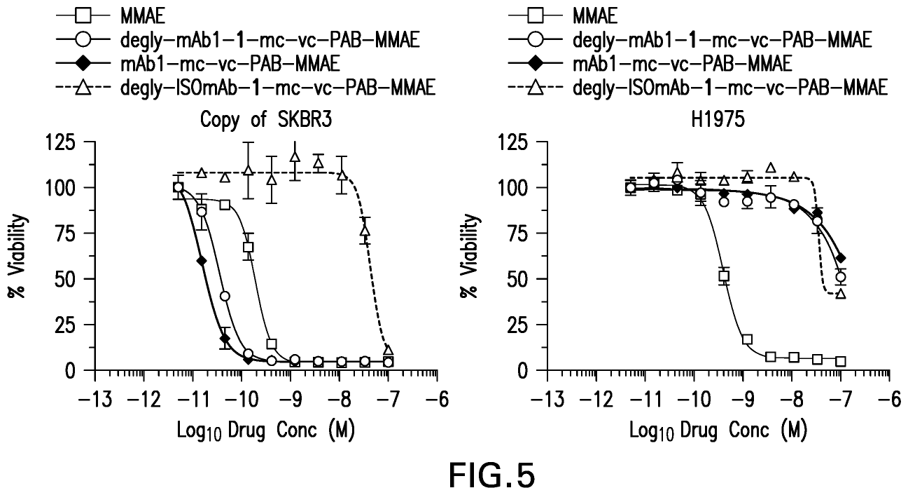

SKBR3 (Her2+) cells were seeded in 96 well plates at 8000 cells per well in complete growth media and grown overnight. For cell viability curves, serially diluted conjugates or naked payloads were added to the cells at final concentrations ranging from 100 nM to 5 pM and incubated for 3 days. NCI H1975 (Her2−) cells were run as negative controls using similar conditions. To measure viability, cells were incubated with CCK8 (Dojindo) for the final 2 hours and the absorbance at 450 nm (OD₄₅₀) was determined on a Victor (Perkin Elmer). Background OD₄₅₀ levels determined from digitonin (40 nM) treated cells were subtracted from all wells and viability is expressed as a percentage of the untreated controls. IC₅₀ values were determined from a four-parameter logistic equation over a 10-point response curve (GraphPad Prism). The IC₅₀ for degly-mAb1-1-mc-vc-PAB-MMAE was 0.035 nM. The interchain disulfide conjugates had similar IC₅₀ values as the transglutaminase site-specific selenocystamine conjugates. This indicates that the selenocystamine conjugations had no effect on the function of the antibody drug conjugate. IC₅₀ values are corrected for payload equivalents and the results of the cell viability are shown in Table 4 and FIG. 5. For comparison, the mc-vc-PAB-MMAE interchain disulfide conjugated molecules were produced according to Doronina, S. O. et al., Nat. Biotechnol., 2003, 21, 778.

TABLE 4

| Cell Type | ADC or Payload | IC₅₀ (nM) | % Kill |
|---|---|---|---|
| SK-BR-3 | MMAE (payload) | 0.191 | 93.6 |
| SK-BR-3 | mAb1-mc-vc-PAB-MMAE | 0.016 | 100 |
| SK-BR-3 | degly-mAb1-1-mc-vc-PAB-MMAE | 0.035 | 100 |
| SK-BR-3 | degly-ISOmAb-1-mc-vc-PAB-MMAE | >100 | — |
| NCI | MMAE (payload) | 0.405 | 100 |
| H1975 | | | |
| NCI | mAb1-mc-vc-PAB-MMAE | >100 | — |
| H1975 | | | |
| NCI | degly-mAb1-1-mc-vc-PAB-MMAE | >100 | — |
| H1975 | | | |
| NCI | degly-ISOmAb-1-mc-vc-PAB-MMAE | >100 | — |
| H1975 | | | |

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula I:
Z-Gln-NH-L-Se-L¹-R
or a pharmaceutically acceptable salt thereof, wherein:
Z is an antigen-binding domain;
NH is the side chain NH of the Gln;
L and L¹ are the same or different and are each a linker; and
R is a payload.

2. The compound of claim 1, wherein Z is an antibody or antigen-binding fragment thereof.

3. The compound of claim 1, wherein Z is an antibody.

4. The compound of claim 1, wherein Z is an N297Q mutant antibody.

5. The compound of claim 1, wherein Z is an antibody that has one or more engineered LLQG, LLQGG, LLQLLQG, LLQYQG, LLQGA, LLQGSG, SLLQG, LQG, LLQLQ, LLQLLQ, LLQGR, LLQYQGA, LQGG, LGQG or LLQLLQGA sites.

6. The compound of claim 1, wherein Gln is Gln295 of an antibody, Gln297 of an N297Q mutant antibody and/or a Gln of an engineered LLQG, LLQGG, LLQLLQG, LLQYQG, LLQGA, LLQGSG, SLLQG, LQG, LLQLQ, LLQLLQ, LLQGR, LLQYQGA, LQGG, LGQG or LLQLLQGA site.

7. The compound of claim 1, wherein Gln is Gln295 of an antibody.

8. The compound of claim 1, wherein L is alkylene, alkenylene, cycloalkylene or arylene, or any combination thereof.

9. The compound of claim 1, wherein L¹ comprises a moiety cleavable by a lysosomal enzyme.

10. The compound of claim 1, wherein L¹ comprises a spacer.

11. The compound of claim 1, wherein R is a therapeutic agent or an imaging agent.

12. The compound of claim 1, wherein R is a cytotoxic agent.

13. The compound of claim 3, wherein the payload to antibody ratio is 1 to 6.

14. A process for synthesizing the compound of claim 1, comprising:
(a) reacting Z-Gln with (H₂N-L-Se)₂ and a transglutaminase; and
(b) reacting the product of step (a) with a reducing agent at pH less than or equal to 6 to form a reduced diselenide in the presence of L¹-R, wherein L¹ comprises a group that reacts with the reduced diselenide to form a covalent bond, thereby forming Z-Gln-NH-L-Se-L¹-R.

15. The process of claim 14, wherein, when the Gln of Z-Gln is Q295 of an N297 antibody, then prior to step (a) the Z-Gln is reacted with a PNGaseF to deglycosylate N297.

16. A compound prepared by the process of claim 15.

17. A pharmaceutical composition, comprising the compound of claim 1 and a pharmaceutically acceptable carrier.

18. A method of treating or diagnosing disease in a subject, comprising administering to the subject the compound of claim 1.

19. A compound of formula II: 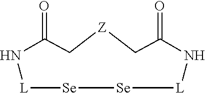
wherein:
Z is an antigen-binding domain;
each 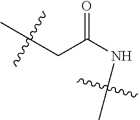 [SMILES: *CC(=O)N*]
is the side chain of Gln; and
L is a linker.

20. The compound of claim 19, having formula IIa: [chemical structure]
